(19) United States
(12) Patent Application Publication
(10) Pub. No.: US 2016/0184199 A1
(43) Pub. Date: Jun. 30, 2016

(54) Cosmetic

(71) Applicant: MITSUBISHI PENCIL COMPANY, LIMITED, Tokyo (JP)

(72) Inventors: Takayuki Hayakawa, Fujioka-shi (JP); Satoshi Sakuma, Fujioka-shi (JP)

(73) Assignee: MITSUBISHI PENCIL COMPANY, LIMITED, Tokyo (JP)

(21) Appl. No.: 14/757,513

(22) Filed: Dec. 23, 2015

(30) Foreign Application Priority Data
Dec. 25, 2014 (JP) 2014-262812
Dec. 17, 2015 (JP) 2015-246367

(51) Int. Cl. A61K 8/29; A61Q 1/10; A61Q 3/02; A61Q 1/02; A61Q 1/06

(52) U.S. Cl. CPC A61K 8/29; A61Q 1/02; A61Q 1/06; A61Q 3/02; A61Q 1/10; A61K 2800/43

(57) ABSTRACT

The cosmetic of the present invention is a cosmetic containing at least titanium oxide having an average particle diameter of 0.2 μm or more and resin fine particles having an average particle diameter of 0.01 to 100 μm.

The above cosmetic is reduced in a content of titanium oxide to decrease troubles brought about by settling thereof in various cosmetics to which whiteness and masking power are required and is endowed with the higher whiteness and the higher masking power in the solid content of the same degree as ever without affecting use feeling of the cosmetic.

This nonprovisional application claims priority under 35 U.S.C. 119(a) on Patent Applications No. 2014-262812 filed in Japan on 25 Dec. 2015 and No. 2015-246367 filed in Japan on 17 Dec. 2015, the entire contents of which are hereby incorporated by reference.

### BACKGROUND OF THE INVENTION

(1) Field of the Invention

The present invention relates to a cosmetic to which whiteness and masking power are required.

(2) Description of Related Art

In various cosmetics such as facial cosmetics and makeup cosmetics including foundations, creams, milky lotions, eye shadows, makeup bases, nail enamels, eyeliners, mascaras, lipsticks, packs, skin lotions, and the like, various white pigments and extender pigments have so far been used in order to exert whiteness and masking power. Cosmetics blended with particularly titanium oxide and zinc oxide out of them can provide a certain degree of masking power.

On the other hand, various spherical resin particles are blended for the same object in a certain case. Known is, for example, a cosmetic containing spherical aggregates (secondary particles) having an average particle diameter of 1 to 100 μm comprising hollow resin spheres having an average particle diameter of 0.01 to 1.0 μm in order to endow covering power which is a defect of resin particles (refer to, for example, JP-A Sho63-119411 (claims, examples and the like)). This is considered to try to form the aggregates of hollow resin particles to complicate diffraction of light, whereby whiteness and masking power grows large.

Also, known is a suntan cosmetic blended with silicon dioxide-coated fine particle titanium oxide and poly(methylhydrosiloxane)-coated fine particle zinc oxide (refer to, for example, JP-A 2001-240524 (claims, examples and the like)).

The respective particles described in the above document are dispersed respectively in different phases of an oil phase and an aqueous phase by about 3% by mass each, and stability and usability (spreading state) are controlled by blending them. Also, it is disclosed in Example 5 of the above document that polyethylene fine particles having an average particle diameter of 10 μm in addition to the respective particles described above are blended as well in 6%, and it is shown that flatness and glossiness of the coated surface are improved by'blending them.

However, a large amount of titanium oxide has to be blended in the respective cosmetics disclosed in the patent documents described above in order to exert whiteness and masking power which are required, and involved therein are the problems that a trouble in which titanium oxide having a large specific gravity settles down and that use feeling of the cosmetic is notably deteriorate. Also, the existing state is that discontent on whiteness and masking power which are inferior only with the resin particles is involved therein.

### SUMMARY OF THE INVENTION

In light of the problems on the conventional technologies and the existing state each described above, an object of the present invention is to provide a cosmetic which is reduced in a content of titanium oxide by suitably combining titanium oxide having specific physical properties with specific resin particles to decrease troubles brought about by settling thereof and which is further enhanced in whiteness and masking power more than those prepared by using titanium oxide alone without affecting a use feeling of the cosmetic.

Intense investigations repeated by the present inventors have resulted in finding that obtained is a cosmetic which is blended with at least titanium oxide having specific physical properties and specific resin particles to thereby enhance whiteness and masking power in the same solid content more than those prepared by using titanium oxide alone due to the interaction of the two components without affecting a use feeling of the cosmetic. Thus, they have come to complete the present invention.

That is, the present invention resides in the following items (1) to (3).
(1) A cosmetic containing at least titanium oxide having an average particle diameter of 0.2 μm or more and resin fine particles having an average particle diameter of 0.01 to 100 μm.
(2) The cosmetic as described in the above item (1), wherein an L [symbol] value of a dried coating film of the cosmetic prepared by replacing a part of the titanium oxide with the resin fine particles shows a higher value than an L [symbol] value of a dried coating film of the cosmetic which is not blended with the resin fine particles and which is prepared by using the titanium oxide alone.
(3) The cosmetic as described in the above item (1) or (2), wherein a viscosity at a shear rate of 3.83 (s⁻¹) and 25° C. is 10 mP·s or more.

According to the present invention, provided is a cosmetic which is provided with whiteness and masking power required to various cosmetics and which is reduced in a content of titanium oxide to decrease troubles brought about by settling thereof and is endowed with the higher whiteness and the higher masking power in the solid contents of the same degree as ever without affecting a use feeling of the cosmetic.

Further, according to the present invention, provided is a cosmetic improved in the rate of drying, compared to the case in which titanium oxide alone is blended without resin fine particles blended in the present invention. The rate of drying is improved better if the resin fine particles have a hollow structure.

As a result thereof, the cosmetic of the present invention can suitably be applied to various cosmetics such as foundations, manicures, milky lotions, creams, packs, lipsticks, and the like to which whiteness and masking power are required.

### DETAILED DESCRIPTION OF THE INVENTION

The embodiments of the present invention shall be explained below in detail.

The cosmetic of the present invention is characterized by containing at least titanium oxide having an average particle diameter of 0.2 μm or more and resin fine particles having an average particle diameter of 0.01 to 100 μm.

The titanium oxide can be used in the present invention without being specifically restricted as long as it has an average particle diameter of 0.2 μm or more.

If the titanium oxide used has an average particle diameter of 0.2 μm or more, it can endow various cosmetics with whiteness and masking power, and it has preferably an average particle diameter of 1 μm or less in terms of whiteness and masking power. The titanium oxide having an average particle diameter of less than 0.2 μm is inferior in whiteness and masking power, and therefore it is not preferred.

In the present invention, the “average particle diameter” means a primary average particle diameter, and it is a value measured based on an electron micrograph.

The titanium oxide which can be used includes, for example, commercially available Tipaque CR-50 (average particle diameter: 0.25 μm, manufactured by Ishihara Sangyo Kaisha, Ltd.), MP-1133 (average particle diameter: 0.25 μm, manufactured by TAYCA CORPORATION), MKR-1 (average particle diameter: 0.2 μm, manufactured by Sakai Chemical Industry Co., Ltd.), and the like. They can be used alone or in a mixture of two or more kinds thereof.

A content of the above titanium oxide shall be varied due to the relationship thereof with resin fine particles described later, and it is preferably 1 to 40% by mass (hereinafter referred to merely as “%”), more preferably 5 to 30% based on a whole amount of the cosmetic.

If a content of the above titanium oxide is less than 1%, the cosmetic is inferior in whiteness and masking power, and on the other hand, if it exceeds 40%, the cosmetic is deteriorated in fluidity and use feeling, so that it is not preferred.

The resin fine particles can be used in the present invention without being specifically restricted as long as they have an average particle diameter of 0.01 to 100 μm.

If the resin fine particles used have an average particle diameter falling in a range of 0.01 to 100 μm, a content of the titanium oxide is reduced due to interaction with the titanium oxide having an average particle diameter of 0.2 μm or more to decrease troubles brought about by settling thereof, and the higher whiteness and the higher masking power than those prepared by using the titanium oxide alone can be endowed to a high extent in the same solid contents without affecting use feeling of various cosmetics. The resin fine particles having an average particle diameter of 0.1 to 1 μm are more preferred. If the resin fine particles used have an average particle diameter of less than 0.01 μm or exceeding 100 μm, the effects of the present invention cannot be exerted, and the cosmetic is short of masking power and deteriorated in finishing, so that it is not preferred.

The resin fine particles which can be used include, for example, (styrene/acrylate) copolymers, (acrylate/maleic acid/styrene) copolymers, polymethyl methacrylate, methyl methacrylate crosspolymers, and the like. They can be used alone or in a mixture of two or more kinds thereof.

The (styrene/acrylate) copolymers and the (acrylate/maleic acid/styrene) copolymers are preferably used from the viewpoint of endowing the cosmetic with the higher whiteness and the higher masking power to a high degree due to interaction with the titanium oxide.

The above resin fine particles are added preferably in the forms of dispersion liquid of an aqueous dispersion and a solvent dispersion from the viewpoints of manufacturability, workability and efficiency of the cosmetic.

The resin fine particles which can specifically be used includes, for example, commercially available OPULYN 305 Opacifier (average particle diameter: 0.3 μm, (acrylate/maleic acid/styrene) copolymer dispersion, manufactured by The Dow Chemical Company), ROHMSPHERE PGL (average particle diameter: 0.35 μm, (styrene/acrylate) copolymer dispersion, manufactured by The Dow Chemical Company), ROHMSPHERE Powder (average particle diameter: 0.35 μm, (styrene/acrylate) copolymer, manufactured by The Dow Chemical Company), MP-2200 (average particle diameter: 0.3 to 0.5 μm, polymethyl methacrylate, manufactured by Soken Chemical & Engineering Co., Lt.), and the like. They can be used alone or in a mixture of two or more kinds thereof.

A content (solid content) of the above resin fine particles is preferably 1 to 50%, more preferably 5 to 40% based on a whole amount of the cosmetic from the viewpoint of exerting the effects of the present invention.

If a content of the above resin fine particles is less than 1%, the effects of the present invention cannot be exerted, and on the other hand, if it exceeds 50%, the cosmetic is deteriorated in fluidity, so that it is not preferred.

The cosmetic of the present invention contains the titanium oxide and the resin fine particles each described above and can further contain a film-forming resin, a thickener and a colorant from the viewpoint of the suitable blend composition.

Film-forming resins used for various cosmetics can be used as the usable film-forming resin without being specifically restricted, and it includes, for example, acrylic acid resin emulsions, (alkyl acrylate/octylacrylamide) copolymers which are alkali-soluble acryl resins, (styrene/acrylate) copolymers, vinyl acetate resins, and the like. They can be used alone or in a mixture of two or more kinds thereof.

A content (solid content) of the above film-forming resins is varied depending on the uses of the cosmetic, and it is preferably 0.1 to 30%, more preferably 1 to 20% based on a whole amount of the cosmetic.

Organic pigments, inorganic pigments, pearl pigments, other bright pigments and dyes which are used usually for cosmetics can be used as the usable colorant.

Capable of being listed as the organic pigments are Blue No. 1 Al lake, Red No. 202, Red No. 226, No. 228, Blue No. 404, Red No. 220, Yellow No. 401, Yellow No. 205, Blue No. 201, Blue No. 204, Yellow No. 4 Al lake, Yellow No. 203 Al lake, Red No. 104 Al lake, and the like; capable of being listed as the inorganic pigments are carbon black, Prussian blue, red iron oxide, yellow iron oxide, black iron oxide, titanium oxide, and the like; and capable of being listed as the pearl pigments are mica, mica titanium, carmine-coated mica titanium, carmine.Prussian blue-coated mica titanium, black iron oxide-coated mica titanium, black iron oxide.carmine-coated mica titanium, black iron oxide.Prussian blue-coated mica titanium, Prussian blue-coated mica titanium, red iron oxide-coated mica, red iron oxide-coated mica titanium, red iron oxide.carmine-coated mica titanium, red iron oxide.black iron oxide-coated mica titanium, red iron oxide.Prussian blue-coated mica titanium, red iron oxide.black iron oxide.Prussian blue-coated mica titanium, and the like.

Capable of being listed as the dyes are statutory dyes and other basic dyes such as Red No. 104, Yellow No. 4, Yellow No. 5, Green No. 3, Blue No. 1, Blue No. 2, Red No. 227, Red No. 230, Yellow No. 202, Green No. 201, Green No. 204, Blue No. 205, Red No. 401, Red No. 504, Orange No. 402, Yellow No. 403, Yellow No. 407, Green No. 401, Purple No. 401, Black No. 401, and the like.

In the present invention, the dyes are preferably used from the viewpoint of making good use of high whiteness and high masking power.

A content of the above colorants is varied depending on the uses of the cosmetic, and it is preferably 0.001 to 10%, more preferably 0.01 to 1% based on a whole amount of the cosmetic.

Thickeners usually used for cosmetics can be used as the usable thickener.

The thickener includes, for example, hydroxyethyl cellulose, microcrystalline cellulose, xanthane gum, gellan gum, guar gum, hydroxypropylguar gum, sclerotium gum, carbomer, and the like. They can be used alone or in a mixture of two or more kinds thereof.

A content of the above thickeners is varied depending on the uses of the cosmetic, and it is preferably 0.001 to 10%, more preferably 0.01 to 1% based on a whole amount of the cosmetic from the viewpoint of controlling viscosity of the cosmetic to a suitable range as described later.

The cosmetic of the present invention can contain water (refined water, distilled water, ion-exchanged water, purified water and the like) as the balance, and it can suitably be further blended, if necessary, with a water-soluble organic solvent for the purpose of controlling the drying property, other optional components, for example, a pH controller, a neutralizing agent, a UV absorber, a UV scattering agent, a wax, a surfactant, a moisturizing agent, a perfume, an antioxidant, an antiseptic agent, a metal ion masking agent, a defoaming agent, various extracts and the like, and other various components.

The organic solvent which can be used includes, for example, alcohols such as ethanol, phenoxyethanol, isopropanol and the like, glycols such as 1,3-butylene glycol, pentylene glycol and the like, and glycerins such as glycerin, ethylhexyl glycerin and the like.

A content of the above organic solvents is 0 to 20%, preferably 0.1 to 20% based on a whole amount of the cosmetic in order to obtain the suitable drying property.

A viscosity of the cosmetic of the present invention is varied depending on the uses of the cosmetic, and a viscosity at a shear rate of 3.83 (s⁻¹) and 25° C. is controlled to preferably 10 (mP·s) or more, more preferably 20 to 10000 (mP·s) and particularly preferably 25 to 1000 (mP·s).

If the above viscosity is less than 10 (mP·s), the titanium oxide forms a hard cake, and the targeted performance is not obtained in a certain case.

The viscosity ranges described above can be controlled by effectively combining the titanium oxide, the resin fine particles, water and the like and using, if necessary, the thickener.

In the cosmetic of the present invention, an L [symbol] value of a dried coating film of the cosmetic (cosmetic of the present invention) prepared by replacing a part of the titanium oxide with the resin fine particles (hereinafter referred to as “an L2 [symbol] value”) shows preferably a higher value than an L [symbol] value of a dried coating film of the cosmetic which is not blended with the resin fine particles described above and which is prepared by using the titanium oxide alone (hereinafter referred to as “an L1 [symbol] value”) in terms of the whiteness and the masking power (L1 [symbol] value<L2 [symbol] value).

The above L [symbol] value shows a brightness in an L*a*b* color system, and in the present invention (including examples described later), it is a brightness (L [symbol] value) measured by means of a spectral colorimeter MSC-IS-2B (manufactured by Suga Test Instruments Co., Ltd.).

“L2 [symbol] value−L1 [symbol] value” falls more preferably in a range of 0.01 to 50 in terms of the whiteness.

The cosmetic of the present invention can be produced by ordinary methods according to the forms of the intended various cosmetics.

The forms of the cosmetic in the present invention are optional and may be any forms of a solution base, a soluble base, an emulsion base, a powder dispersion base, a water-oil two phase base, a water-oil-powder three phase base, gel, mist, spray, mousse, roll-on, and the like.

Also, the product forms of the cosmetic are optional as well and can be applied to, for example, facial cosmetics such as skin lotions, milky lotions, creams, packs and the like, makeup cosmetics such as foundations, lipsticks, eyeliners, mascaras, eye shadows and the like, sunscreen cosmetics (sunscreen agents), body cosmetics, manicures (nail enamels), and the like. It is, for example, filled into various applicators, etc. and used.

It can suitably be applied particularly to foundations, manicures, milky lotions, creams, packs, lipsticks, temporary hair dyes, body arts, eye shadows and eyeliners to which whiteness and masking power are required in various cosmetics.

The cosmetic of the present invention thus constituted contains at least the titanium oxide having an average particle diameter of 0.2 μm or more and the resin fine particles having an average particle diameter of 0.01 to 100 μm, and obtained is a cosmetic which is reduced in a content of the titanium oxide to decrease troubles brought about by settling thereof in various cosmetics requiring whiteness and masking power and which is endowed with the higher whiteness and the higher masking power in the solid content of the same degree as ever without affecting use feeling of the cosmetic.

### EXAMPLES

Next, the present invention shall be explained in further details with reference to examples and comparative examples, but the present invention shall not be restricted by the examples and the like shown below. Examples 1 to 9 and Comparative Examples 1 to 5 Examples 1 to 8 and Comparative Examples 1 to 4:

The components were mixed and dispersed in blend formulations (whole amount: 100%) shown in the following Table 1 by means of a disper to prepare the respective cosmetics (manicures; nail enamels).

### Example 9 and Comparative Example 5

The components were mixed and dispersed in blend formulations (whole amount: 100%) shown in the following Table 2 by means of a homomixer to prepare the respective cosmetics (foundations).

The respective cosmetics (manicures and foundations) obtained in Examples 1 to 9 and Comparative Examples 1 to 5 were used to measure the respective viscosities at a shear rate of 3.83 (s⁻¹) and 25° C. by the following measuring method and measure an L1 [symbol] value of a dried coating film of the cosmetic which is not blended with the resin fine particles and which is prepared by using the titanium oxide alone and an L2 [symbol] value of a dried coating film of the cosmetic of the present invention by the following respective measuring methods, and the masking powers thereof were evaluated.

The results thereof are shown in the following Tables 1 and 2.

### Measuring Method of Viscosity:

The respective cosmetics for eyelash decoration were used to measure a viscosity at the prescribed shear rate and a temperature of 25° C. by means of a cone-plate type viscometer (EHD viscometer out of TV-30 viscometers, standard cone-plate, manufactured by Tokimec, Inc.). Measuring method of respective L1 [symbol] values of dried coating films of the cosmetics:

An L1 [symbol] value of a dried coating film of the cosmetic which is not blended with the resin fine particles and which is prepared by using the titanium oxide alone and an L2 [symbol] value of a dried coating film of the cosmetic of the present invention were measured by the following measuring method.

The cosmetic was coated in an even thickness and naturally dried, and then the coating film was used to measure the respective L1 [symbol] values by means of a spectral colorimeter.

### Evaluating Method of Masking Power:

The respective cosmetics were coated in an even thickness on a masking rate test paper and naturally dried, and then the coating films were visually observed to evaluate the masking powers according to the following respective evaluation criteria.

### Evaluation Criteria of Masking Power:

◯: boundary between black and white parts is not observed
Δ: boundary between black and white parts is a little observed
×: boundary between black and white parts is clearly observed

### Evaluation Method of the Rate of Drying:

Each cosmetic is applied in a uniform thickness on a masking power test paper, and 3 minutes later, stroked softly with a cotton applicator.

The rate of drying was evaluated under the following criteria.

### The Criteria for the Rate of Drying:

◯: the cosmetic is not put on the cotton applicator at all
Δ: the cosmetic is not put on the cotton applicator, but the layer of the cosmetic deforms to an extent
×: the cosmetic is put on the cotton applicator, and the layer of applied cosmetic deforms

TABLE 1

| | | | Example | | | | | | | |
|---|---|---|---|---|---|---|---|---|---|---|
| Blend composition (manicure, nail art) | | Product example | 1 | 2 | 3 | 4 | 5 | 6 | 7 | 8 |
| Titanium | Titanium oxide | Tipaque CR-50 [symbol] 1 | 13 | 5 | 13 | 13 | 5 | 2.5 | 13 | 13 |
| oxide 1 | | | | | | | | | | |
| Titanium | Fine particle titanium | TTO-55(A) [symbol] 2 | | | | | | | | |
| oxide 2 | oxide | | | | | | | | | |
| Resin particle | (Acrylate/maleic acid | OPULYN 305 | 16 | | 16 | 16 | | 18.7 | 16 | |
| dispersion 1 | PEG-10/styrene) | Opacifier [symbol] 3 | | | | | | | | |
| | copolymer | | | | | | | | | |
| Resin particle | (Styrene/acrylate) | ROHMSPHERE | | 33 | | | | | | |
| dispersion 2 | copolymer | PGL [symbol] 4 | | | | | | | | |
| Resin particle | (Styrene/acrylate) | DAITOSOL PLUS | | | | | | | | 10 |
| dispersion 3 | copolymer | MT-PE [symbol] 20 | | | | | | | | |
| Resin particle 1 | (Styrene/acrylate) | ROHMSPHERE | | | | | 12 | | | |
| | copolymer | Powder [symbol] 5 | | | | | | | | |
| Resin particle 2 | (Styrene/acrylate) | Manufactured by | | | | | | | | |
| | copolymer | Mitsubishi Pencil | | | | | | | | |
| | | Co., Ltd., particle | | | | | | | | |
| | | diameter: 200 μm | | | | | | | | |
| Film-forming | Acryl resin emulsion | Yodosol GH34F [symbol] 8 | | | 10 | | | | | |
| agent 1 | | | | | | | | | | |
| Film-forming | Alkali-soluble acryl | AMPHOMER HC [symbol] 9 | 1.82 | 2.15 | | 2 | 2.15 | 2.15 | 2 | 1.8 |
| agent 2 | resin | | | | | | | | | |
| Thickener 1 | Microcrystalline | Ceolus RC-N81 [symbol] 6 | | | 1.6 | | | | | |
| | cellulose | | | | | | | | | |
| Thickener 2 | Xanthane gum | KELTROL [symbol] 7 | 0.6 | 0.6 | | 0.6 | 0.6 | 0.6 | | |
| Thickener 3 | Succinoglycan | RHEOZAN SH [symbol] 21 | | | | | | | | 0.4 |
| Colorant 1 | Red No. 202 | Red No. 202 | | | | 0.5 | | | | |
| Colorant 2 | Yellow No. 4 | Yellow No. 4 | | | | 3 | | | | |
| Antiseptic | Methylparaben, | Methylparaben, | 0.3 | 0.3 | 0.3 | 0.3 | 0.3 | 0.3 | 0.3 | 0.3 |
| agent | ethylparaben | ethylparaben | | | | | | | | |
| Solvent 1 | 1,3-Butylene glycol | 1,3-Butylene glycol | 7 | 7 | 7 | 7 | 7 | 7 | 7 | 6.5 |
| Solvent 2 | Phenoxyethanol | Phenoxyethanol | 0.6 | 0.6 | 0.6 | 0.6 | 0.6 | 0.6 | 0.6 | 0.6 |
| Solvent 3 | Ethylhexyl glycerin | Sensiva SC50JP [symbol] 10 | 1 | 1 | 1 | 1 | 1 | 1 | 1 | 1 |
| Solvent 4 | Water | Refined water | bal- | bal- | bal- | bal- | bal- | bal- | bal- | bal- |
| | | | ance | ance | ance | ance | ance | ance | ance | ance |
| Evaluation | L* value (L1* value) | | 69 | 69 | 69 | 69 | 67 | 50 | 69 | 69 |
| | of cosmetic prepared | | | | | | | | | |
| | by using titanium | | | | | | | | | |
| | oxide alone | | | | | | | | | |
| | L* value (L2 *value) | | 75 | 75 | 75 | 75 | 75 | 58 | 75 | 72 |
| | of cosmetic | | | | | | | | | |
| | Viscosity (3.83 s⁻¹, | | 1700 | 1800 | 100 | 1600 | 1600 | 1700 | 3 | 1500 |
| | mPa · s) | | | | | | | | | |
| | Masking power | | ∘ | ∘ | ∘ | ∘ | ∘ | Δ | Δ | ∘ |
| | The rate of drying | | ∘ | ∘ | ∘ | ∘ | ∘ | ∘ | ∘ | ∘ |

| | | | Comparative Example | | | |
|---|---|---|---|---|---|---|
| Blend composition (manicure, nail art) | | Product example | 1 | 2 | 3 | 4 |
| Titanium oxide 1 | Titanium oxide | Tipaque CR-50 [symbol] 1 | | 5 | 18 | |
| Titanium oxide 2 | Fine particle titanium | TTO-55(A) [symbol] 2 | 5 | | | |
| | oxide | | | | | |
| Resin particle | (Acrylate/maleic acid | OPULYN 305 | | | | |
| dispersion 1 | PEG-10/styrene) | Opacifier [symbol] 3 | | | | |
| | copolymer | | | | | |
| Resin particle | (Styrene/acrylate) | ROHMSPHERE | 33 | | | 45 |
| dispersion 2 | copolymer | PGL [symbol] 4 | | | | |
| Resin particle | (Styrene/acrylate) | DAITOSOL PLUS | | | | |
| dispersion 3 | copolymer | MT-PE [symbol] 20 | | | | |
| Resin particle 1 | (Styrene/acrylate) | ROHMSPHERE | | | | |
| | copolymer | Powder [symbol] 5 | | | | |
| Resin particle 2 | (Styrene/acrylate) | Manufactured by | | 12 | | |
| | copolymer | Mitsubishi Pencil | | | | |
| | | Co., Ltd., particle | | | | |
| | | diameter: 200 μm | | | | |
| Film-forming agent 1 | Acryl resin emulsion | Yodosol GH34F [symbol] 8 | | | | |
| Film-forming agent 2 | Alkali-soluble acryl resin | AMPHOMER HC [symbol] 9 | 2.15 | 2.15 | 2 | 2 |
| Thickener 1 | Microcrystalline cellulose | Ceolus RC-N81 [symbol] 6 | | | | |
| Thickener 2 | Xanthane gum | KELTROL [symbol] 7 | 0.6 | 0.6 | 0.6 | 0.6 |
| Thickener 3 | Succinoglycan | RHEOZAN SH [symbol] 21 | | | | |
| Colorant 1 | Red No. 202 | Red No. 202 | | | | |
| Colorant 2 | Yellow No. 4 | Yellow No. 4 | | | | |
| Antiseptic agent | Methylparaben, | Methylparaben, | 0.3 | 0.3 | 0.3 | 0.3 |
| | ethylparaben | ethylparaben | | | | |
| Solvent 1 | 1,3-Butylene glycol | 1,3-Butylene glycol | 7 | 7 | 7 | 7 |
| Solvent 2 | Phenoxyethanol | Phenoxyethanol | 0.6 | 0.6 | 0.6 | 0.6 |
| Solvent 3 | Ethylhexyl glycerin | Sensiva SC50JP [symbol] 10 | 1 | 1 | 1 | 1 |
| Solvent 4 | Water | Refined water | bal- | bal- | bal- | bal- |
| | | | ance | ance | ance | ance |
| Evaluation | L* value (L1* value) | | 30 | 59 | 69 | 69 |
| | of cosmetic prepared | | | | | |
| | by using titanium | | | | | |
| | oxide alone | | | | | |
| | L* value (L2 *value) | | 45 | 60 | 69 | 50 |
| | of cosmetic | | | | | |
| | Viscosity (3.83 s⁻¹, | | 1600 | 1600 | 1600 | 1600 |
| | mPa · s) | | | | | |
| | Masking power | | x | Δ | Δ | x |
| | The rate of drying | | ∘ | ∘ | x | ∘ |

[symbol]

1: average particle diameter: 250 μm, manufactured by Ishihara Sangyo Kaisha, Ltd. [symbol]
2: average particle diameter: 10 to 30 μm, manufactured by Ishihara Sangyo Kaisha, Ltd. [symbol]
3: average particle diameter: 300 μm, solid content: 40%, manufactured by The Dow Chemical Company [symbol]
4: average particle diameter: 350 μm, solid content: 26%, manufactured by The Dow Chemical Company [symbol]
5: average particle diameter: 350 μm, manufactured by The Dow Chemical Company [symbol]
6: manufactured by Asahi Kasei Chemicals Corporation [symbol]
7: manufactured by Sansho Co., Ltd. [symbol]
8: alkyl acrylate copolymer ammonium, solid content: 42%, manufactured by Akzo Nobel N.V. [symbol]
9: (alkyl acrylate/octylacrylamide) copolymer, manufactured by Akzo Nobel N.V. [symbol]
10: manufactured by Schülke & Mayr [symbol]
20: average particle diameter: 1 μm, solid content: 28%, manufactured by Daito Kasei Kogyo Co., Ltd. [symbol]
21: manufactured by Solvay Nicca, Ltd.

TABLE 2

| | | | | Comparative |
|---|---|---|---|---|
| | | | Example | Example |
| Blend composition (foundation) | | Product example | 9 | 5 |
| Surfactant | Polyglyceryl sesquiisostearate-2 | Hostacerin ® DGI [symbol] 11 | 2.4 | 2.4 |
| Titanium oxide | Titanium oxide (CI 77891) | Unipure White LC ® 981 [symbol] 12 | 3.5 | 7 |
| Colorant | Iron oxide (CI 77491 + CI 77492 + | Unipure Brown LC ® 887 [symbol] 13 | 8 | 8 |
| | CI 77499) | | | |
| Solvent | Cyclopentasiloxane, capryl | BELSIL ® SPG 128 VP [symbol] 14 | 12 | 12 |
| | dimethiconeethoxyglucoside | | | |
| Stabilizer | beeswax | Deodorized and refined | 2.7 | 2.7 |
| | | beeswax high acid [symbol] 15 | | |
| Extender | talc | Talc MSA [symbol] 16 | 5 | 5 |
| pH controller | Sodium chloride | Sodium Chloride | 2 | 2 |
| Resin fine particle | (Acrylate/maleic acid PEG-10/styrene) | OPULYN 305 Opacifier [symbol] 3 | 8.75 | |
| dispersion | copolymer | | | |
| Solvent | Silicone | BELSIL ® DM 0.65 [symbol] 17 | 15 | 15 |
| Antiseptic agent | Methylchloroisothiazolinone, | Kathon ™ CG [symbol] 18 | 0.06 | 0.06 |
| | methylisothiazolinone | | | |
| Film-forming agent | (Vinyl dimethyl/trimethyl | BELSIL ® REG 1100 [symbol] 19 | 2 | 2 |
| | siloxysilicate) crosspolymer | | | |
| Solvent | Water | Refined water | balance | balance |
| Evaluation | L* value (L1* value) of cosmetic prepared | | 60 | 60 |
| | by using titanium oxide alone | | | |
| | L* value (L2* value) of cosmetic | | 65 | 60 |
| | Viscosity (3.83 s⁻¹, mPa · s) | | 2000 | 2000 |
| | Masking power | | ∘ | ∘ |
| | The rate of drying | | ∘ | × |

[symbol]

3: average particle diameter: 0.3 μm, solid content: 40 %, manufactured by The Dow Chemical Company [symbol]
11: manufactured by Clariant (Japan) K. K. [symbol]
12: average particle diameter: 0.3 μm, manufactured by Sensient Technologies Corporation [symbol]
13: manufactured by Sensient Technologies Corporation [symbol]
14: manufactured by Wacker Asahikasei Silicone Co., Ltd. [symbol]
15: manufactured by CERARICA NODA Co., Ltd. [symbol]
16: manufactured by Daito Kasei Kogyo Co., Ltd. [symbol]
17: manufactured by Wacker Asahikasei Silicone Co., Ltd. [symbol]
18: manufactured by The Dow Chemical Company [symbol]
19: manufactured by Wacker Asahikasei Silicone Co., Ltd.

As apparent from the results summarized in Table 1 and Table 2 described above, it has become clear that the cosmetics of the present invention obtained in Examples 1 to 8, which are endowed with the higher whiteness and the higher masking power without affecting use feeling of the cosmetics, compared to the cosmetics prepared in Comparative Examples 1 to 5 falling outside the scope of the present invention.

What is claimed is:

1. A cosmetic containing at least titanium oxide having an average particle diameter of 0.2 μm or more and resin fine particles having an average particle diameter of 0.01 to 100 μm.

2. The cosmetic as described in claim 1, wherein an L [symbol] value of a dried coating film of the cosmetic prepared by replacing a part of the titanium oxide with the resin fine particles shows a higher value than an L [symbol] value of a dried coating film of the cosmetic which is not blended with the resin fine particles and which is prepared by using the titanium oxide alone.

3. The cosmetic as described in claim 1, wherein a viscosity at a shear rate of 3.83 (s⁻¹) and 25° C. is 10 mP·s or more.
